(19) United States
(12) Patent Application Publication
(10) Pub. No.: US 2009/0099612 A1
(43) Pub. Date: Apr. 16, 2009

(54) ELECTRICAL CONDUCTOR HAVING A BIOERODIBLE COATING

(76) Inventor: Julie S. Armstrong, Glendale, CA (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/251,014

(22) Filed: Oct. 14, 2008

Related U.S. Application Data
(60) Provisional application No. 60/980,041, filed on Oct. 15, 2007.

(51) Int. Cl. A61N 1/36; A61N 1/05

(52) U.S. Cl. 607/3; 607/116

(57) ABSTRACT

An apparatus includes an elongate member having a proximal end portion and a distal end portion. The proximal end portion is configured to receive an electrical current from a current source. The elongate member is configured to transmit the electrical current from the proximal end portion to the distal end portion. At least a portion of the elongate member is configured to be disposed within a body of a patient. An electrode is coupled to the distal end portion of the elongate member. The electrode is configured to transmit a portion of the electrical current from the distal end portion of the elongate member to a target bodily tissue. A coating is disposed on at least a portion of the elongate member. The coating is formulated to release at least one of a therapeutic agent, a conductive agent, and/or an insulative agent into the body of the patient in response to the electrical current being transmitted from the proximal end portion to the distal end portion of the elongate member.

### CROSS-REFERENCE TO RELATED APPLICATIONS

This application claims priority to U.S. Provisional Application Ser. No. 60/980,041, entitled “Electrical Conductor,” filed Oct. 15, 2007, which is incorporated herein by reference in its entirety.

### BACKGROUND

The invention relates generally to a medical device, and specifically to an implanted electrical conductor for routing electrical current to a target bodily tissue in a body of a patient.

Electrical conductors, also referred to as medical leads, are used for various indications, such as for electrical stimulation and/or blocking impulses of a target bodily tissue, delivery of an electrical current to an implanted electrical device, and/or delivery of an electrical current from a medical device to the target bodily tissue. For example, known medical leads can be used in percutaneous electrical nerve stimulation (PENS), which includes transmitting an electrical current from an electrical device disposed outside of the body to a target bodily tissue (e.g., a nerve, a muscle, or the like).

Some known systems for conveying an electrical current between an electrical device disposed outside of a body and a target bodily tissue include a passive electrical conductor and at least one surface electrode. The passive electrical conductor, when placed within the body of a patient, extends from subcutaneous tissue located below a surface electrode to the target bodily tissue. The passive electrical conductor has a pick-up end for receiving the electrical current from the surface electrode such that the electrical current can flow through the conductor, and a stimulating end for delivering electrical current to the target bodily tissue. A surface return electrode can also be positioned on the skin. In use, the system can apply sub-sensational levels of transcutaneous stimulation (i.e., electrical current) that is delivered via the passive electrical conductor to the target bodily tissue. In some such procedures, however, a portion of the electrical current can be attenuated due to the electrical impedance of the skin and/or the surrounding bodily tissue. Thus, only a portion of the electrical current produced by the electrical device is picked up by the electrical conductor and/or received by the target bodily tissue.

Moreover, in some such procedures, it is not uncommon for a patient to experience pain, infection, and/or inflammation due to the implantation of the passive electrical conductor within the patient's bodily tissue. Known treatments for such indications include oral administration of medication and injection of medication. Such known treatments, however, may not accomplish targeted and sustained drug delivery. For example, oral medications are not specific to the area of pain, infection and/or inflammation. Thus, a treatment including oral medications may require an increase in the amount of systemic drug and may cause undesirable side effects. In another example, injection of medication into the affected tissue to locally anesthetize the affected tissue may inhibit testing for optimal placement of the electrical conductor within the body. For example, in some known procedures, a probe may be used during implantation to determine the most efficacious location of an electrical conductor for achieving a desired response. In such procedures, electrical current is applied through the probe to test the response of the target bodily tissue, including the patient's response with respect to the reduction of pain. Thus, use of a local anesthetic to treat or prevent pain, infection, and/or inflammation may inhibit testing for optimal placement of the electrical conductor.

Drug delivery systems are also used for the treatment of pain, infection, and/or inflammation not attributable to implantation of a foreign body. Known drug delivery systems include iontophoresis and intrathecal drug delivery. Systems for intrathecally administering medication usually require implantation of a drug delivery pump in subcutaneous tissue of the patient. Such systems can have high revision and/or infection rates. Further, some patients that would otherwise benefit from such systems are physically incapable of using such systems. For example, some patients lack the level of subcutaneous fat required for the pump to be implanted.

Thus, a need exists for an electrical conductor configured to minimize the attenuation of electrical current within surrounding bodily tissue and/or to increase the amount of electrical current that is transferred to the target bodily tissue. Additionally, a need exists for an implantable device, such as an electrical conductor, configured to deliver a therapeutic agent to bodily tissue surrounding the implanted device during implantation of the device and/or for the minutes, hours, days, weeks, or longer period of time following implantation. A need also exists for a compact drug delivery system suitable for a variety of patients.

### SUMMARY

An apparatus includes an elongate member having a proximal end portion and a distal end portion. The proximal end portion is configured to receive an electrical current from a current source, which can be, for example internal to the body or an external source. The elongate member is configured to transmit the electrical current from the proximal end portion to the distal end portion. At least a portion of the elongate member is configured to be disposed within a body of a patient. An electrode is coupled to the distal end portion of the elongate member. The electrode is configured to transmit a portion of the electrical current from the distal end portion of the elongate member to a target bodily tissue. A coating is disposed on at least a portion of the elongate member. The coating is formulated to release at least one of a therapeutic agent, a conductive agent, and/or an insulative agent into the body of the patient in response to the electrical current being transmitted from the proximal end portion to the distal end portion of the elongate member.

### BRIEF DESCRIPTION OF THE DRAWINGS

FIG. 1 is a schematic illustration of a medical implant according to an embodiment.

FIG. 2 is a side view of an electrical conductor according to an embodiment in a first configuration.

FIG. 3 is a cross-sectional view of the electrical conductor of FIG. 2 taken along line X-X.

FIG. 4 is a side view of a portion of the electrical conductor of FIG. 2 in a second configuration.

FIG. 5 is a side view of a medical implant according to an embodiment.

FIGS. 6 and 7 are side views of a medical implant according to an embodiment implanted within bodily tissue in a first configuration and a second configuration.

FIG. 8 is a side view of a medical implant according to an embodiment.

FIG. 9 is a cross-sectional view of the medical implant of FIG. 8 taken along line Y-Y.

FIG. 10 is a flowchart of a method according to an embodiment.

### DETAILED DESCRIPTION

Apparatus and methods for transmitting an electrical current to a target bodily tissue of a patient are described herein. For example, a medical implant according to an embodiment is configured to receive an electrical input from a stimulator and to transmit the electrical input as an electrical current from a proximal end portion of the medical implant to a distal end portion of the medical implant and from the distal end portion of the medical implant to a target bodily tissue via an electrode. A coating disposed on the elongate member is formulated to release at least one of a therapeutic agent, a conductive agent, and/or an insulative agent into the body of the patient in response to the electrical current being transmitted from the proximal end portion to the distal end portion of the elongate member.

In another example, a medical implant according to an embodiment is configured to receive an electrical input from a stimulator and to transmit the electrical input as an electrical current from a proximal end portion of the medical implant to a distal end portion of the medical implant and from the distal end portion of the medical implant to a target bodily tissue via an electrode. A coating disposed on the elongate member is formulated to release at least one of a conductive agent and/or an insulative agent into the body of the patient.

A method of coating a portion of the medical implant is also described herein. For example, in one embodiment, a polymer is dissolved into a solvent to produce a polymer-solvent solution. A material is disposed in the polymer-solvent solution to produce a polymer-solvent-material solution. The solvent is removed from the polymer-solvent-material solution to produce a polymer-material solution. A portion of the medical implant is coated with the polymer-material solution and the polymer-material solution is solidified on the portion of the medical implant.

A method of releasing a material from a coating on the medical implant into a body of a patient is also described herein. For example, in one embodiment, at least a portion of the medical implant is implanted within a body of a patient. An electrical current is transmitted to the medical implant. The material is released from the coating of the medical implant in response to the transmitting of the electrical current.

As used herein, bodily tissue can include any tissue of a patient suitable for receiving an electrical stimulation. Bodily tissue can include, for example, nervous tissue, such as a nerve, the spinal cord, or another component of the peripheral or central nervous system. In another example, bodily tissue can include muscle tissue, such as, for example, skeletal muscle, smooth muscle, or cardiac muscle. Specifically, bodily tissue can include a group of tissues forming an organ, such as, for example, the skin, lungs, cochlea, heart, bladder, or kidney. In still another example, bodily tissue can include connective tissue, such as, for example, bone or bone-like tissue.

As used in this specification, the words “proximal” and “distal” can refer to the direction closer to and away from, respectively, an operator (e.g., surgeon, physician, nurse, technician, etc.) who would use a medical device or a therapeutic device during a procedure. For example, the end of a medical device first to contact the patient's body would be the distal end, while the opposite end of the medical device (e.g., the end of the medical device being operated by the operator) would be the proximal end of the medical device. Similarly, the end of a medical device implanted the furthest within the patient's body would be the distal end, while the opposite end of the medical device (e.g., the end of the medical device that is implanted the least amount within the body or the end of the medical device that is disposed outside of the body) would be the proximal end.

The medical implant can be configured to treat a variety of medical conditions, including but not limited to acute and/or chronic pain, and/or to activate a motor point. For example, the medical implant can be configured to transmit an electrical current that at least partially activates conduction and/or propagation of action potentials (nerve impulses) along the axons of a target nerve associated with the target bodily tissue. In another example, the medical implant can be configured to transmit to the bodily tissue an electrical current that at least partially blocks the conduction and/or propagation of action potentials along the axons of the target nerve associated with the target bodily tissue.

The medical implant can be configured for percutaneous stimulation of the target bodily tissue. In a treatment or procedure for percutaneous stimulation, for example, the medical implant is configured to transmit an electrical stimulation to the target bodily tissue. In various procedures, the medical implant can be completely or partially implanted within the bodily tissue. In a procedure in which the medical implant is partially implanted within the bodily tissue, for example, a portion of the medical implant is within the body of the patient and a portion of the medical implant extends through the skin such that another portion of the medical implant is external to the body of the patient. For example, percutaneous stimulation can be used to deliver stimulation without activating the skin receptors because the electrical stimulation is transmitted to the medical implant without first passing through the patient's skin.

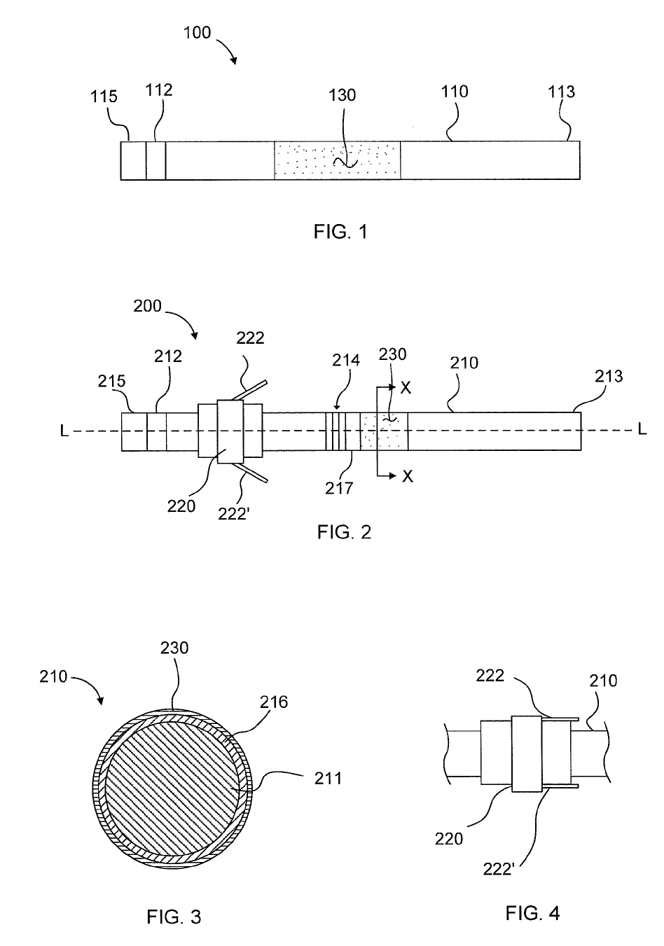

FIG. 1 is a schematic illustration of a medical implant 100 according to an embodiment. The medical implant 100 can be, for example, an electrical conductor or lead. The medical implant 100 includes an elongate member 110, an electrode 112, and a coating 130. At least a portion of the elongate member 110 is configured to be disposed within a body of a patient.

The elongate member 110 includes a proximal end portion 113 and a distal end portion 115. The proximal end portion 113 of the elongate member 110 is configured to receive an electrical current from and/or transmit an electrical current to an external source (not shown). For example, the proximal end portion 113 of the elongate member 110 can be configured to receive the electrical current from a source that is external to the elongate member 110, external to the medical implant 100, and/or external to the body of the patient. The external source can be, for example, a signal processor, a sensor, a stimulator, or the like.

The proximal end portion 113 of the elongate member 110 can be configured to receive an electrical current transmitted to the proximal end portion 113 by any suitable electrical current transmitting mechanism. The transmission of current to an implanted medical implant is described, for example, in U.S. Patent Publication No. 2006/0184211, entitled “Method of Routing Electrical Current to Bodily Tissues via Implanted Passive Conductors,” filed on Aug. 17, 2006, which is incorporated herein by reference in its entirety. In another example, the proximal end portion 113 of the elongate member 110 can be configured to receive an electrical current transmitted to the proximal end portion 113 via an electrode-battery assembly (not shown) configured to transmit the electrical current through bodily tissue proximate to the proximal end portion 113, such as an electrode-battery assembly described in U.S. patent application Ser. No. 12/197,849, entitled “System for Transmitting Electrical Current to a Bodily Tissue,” filed on Aug. 25, 2008, which is incorporated herein by reference in its entirety. In yet another example, the proximal end portion 113 of the elongate member 110 can be configured to receive an electrical current transmitted to the proximal end portion 113 from a stimulator (not shown) external to the body of the patient via a mechanical connector (not shown), as described in U.S. patent application Ser. No. 12/197,849.

The elongate member 110 is configured to transmit the electrical current from the proximal end portion 113 to the distal end portion 115. For example, in some embodiments, the elongate member 110 can include a conductive pathway (not shown) extending from the proximal end portion 113 to the distal end portion 115. In some embodiments, the conductive pathway can include substantially all of the elongate member 110. In other embodiments, the conductive pathway can include only a portion of the elongate member 110 (e.g., a central core).

The electrode 112 is coupled to the distal end portion 115 of the elongate member 110. The electrode 112 is configured to transmit at least a portion of the electrical current from the distal end portion 115 of the elongate member 110 to a target bodily tissue (not shown in FIG. 1). The electrode 112 can be any suitable electrode, such as, for example, a cuff electrode. Although the medical implant 100 is shown as including one electrode 112, in other embodiments, a medical implant can include multiple electrodes.

The coating 130 is disposed on at least a portion of the elongate member 110. The coating 130 is formulated to release at least one of a therapeutic agent, a conductive agent, or an insulative agent into the body of the patient. In some embodiments, for example, the coating can include an antimicrobial agent, an anti-inflammatory agent, a pain-relieving agent, or the like. In another example, the coating can include an electrically conductive medium, electrically conductive particles, or the like. In yet another example, the coating can include an insulative medium, insulative polymer, insulative particles, or the like.

FIGS. 2-4 illustrate an electrical conductor 200 according to an embodiment. The electrical conductor 200 is a passive electrical conductor configured to conduct (or transmit) an electrical current between an external source and a target bodily tissue (not shown) of a patient. The electrical conductor 200 includes an elongate member 210, an electrode 212, and a fixation mechanism 220.

The elongate member 210 has a proximal end portion 213, a distal end portion 215, and a central portion 217 extending between the proximal end portion 213 and the distal end portion 215. The central portion 217 can be referred to, for example, as the lead portion. The proximal end portion 213 of the electrical conductor 200 is configured to be electrically coupled or connected to a stimulator (not shown). For example, the proximal end portion 213 of the electrical conductor 200 can be configured to be electrically coupled to and/or in electrical communication with an electrode-battery assembly (not shown) that is coupled to the stimulator. The proximal end portion 213 of the elongate member 210 is configured to receive at least a portion of the electrical current transmitted from the stimulator. In use, the proximal end portion 213 of the elongate member 210 can be coupled to the body of the patient, e.g., to a portion of the skin of the patient, with an adhesive, a bandage, or the like. In other embodiments, the proximal end portion 213 of the elongate member 210 can be disposed beneath the skin of the patient.

The elongate member 210 is configured to transmit the electrical current from the proximal end portion 213 to the distal end portion 215 of the elongate member 210, such as via the central portion 217. The electrode 212 is configured to transmit at least a portion of the electrical current from the distal end portion 215 of the elongate member 210 to the target bodily tissue. In this manner, the electrical conductor 200 can be used to stimulate the target bodily tissue. For example, the electrical current stimulates the target bodily tissue by at least partially activating and/or blocking the conduction and/or propagation of action potentials or nerve impulses along the axons of nerves associated with the target bodily tissue.

As shown in FIG. 3, the elongate member 210 includes a conductive core 211, an insulative layer 216, and a coating 230. The core 211 is configured to transmit the electrical current from the proximal end portion 213 of the elongate member 210 through the central portion 217 to the distal end portion 215 of the elongate member 210. The elongate member 210 and/or the core 211 can be constructed of any material suitable for transmitting or routing an electrical current within a body of a patient. For example, in some embodiments, the elongate member 210 and/or the core 211 of the electrical conductor 200 is constructed of at least one of a metal wire, carbon fibers, a conductive rubber or other conductive polymer, a conductive salt solution in rubber, or the like. For example, in some embodiments, the elongate member 210 and/or the core 211 can be constructed of multi-stranded, Teflon®-insulated, stainless-steel wire. In another example, the elongate member can be constructed of at least one of MP35N® alloy (a nonmagnetic, nickel-cobalt-chromium-molybdenum alloy) or alloys of platinum and/or iridium.

The insulative layer 216 is disposed on and/or over a portion of the electrical conductor 200, e.g., an outer surface of the core 211 of the elongate member 210, as illustrated in FIG. 3. The insulative layer 216 is configured to minimize and/or reduce an amount of electrical current lost to bodily tissue proximate to the electrical conductor 200 when the electrical current is transmitting through the elongate member 210. Similarly stated, thickness and/or material properties (e.g., resistivity) of the insulative layer 216 are configured to limit attenuation of the electrical current conveyed by the electrical conductor 200. The insulative layer 216 is biocompatible and can be constructed of any suitable material, as described in more detail herein.

The insulative layer 216 of the elongate member 210 is configured to anchor and/or retain the elongate member 210 within the bodily tissue of the patient. As illustrated in FIG. 2, the insulative layer 216 defines a set of grooves each extending transversely to a longitudinal axis L of the electrical conductor 200. Specifically, the set of grooves 214 extends circumferentially about the insulative layer 216 orthogonally to the longitudinal axis L of the electrical conductor 200. The set of grooves 214 is configured to permit movement of the electrical conductor 200 in a forward (or distal) direction, such as during implantation. The set of grooves 214 is configured to engage bodily tissue to prevent movement of the electrical conductor 200 in a backwards (or proximal) direction, such as after insertion into bodily tissue. Thus, the insulative layer 216 is configured to minimize migration of the electrical conductor 200 within the body of the patient. The set of grooves 214 can be formed in the insulative layer 216 by any known means, including cutting and/or etching the grooves in the insulative layer 216. For example, in some embodiments, the set of grooves 214 can be laser etched into the insulative layer 216.

The coating 230 is disposed on an outer surface of the insulative layer 216 of the elongate member 210, as illustrated in FIG. 3. As described in more detail herein, the coating 230 can be formed on or applied to the insulative layer 216 of the elongate member 210 by any suitable means for coating a medical device, including, for example, dip coating, spray coating, chemical bonding, deposition or extrusion. As illustrated in FIG. 2, the coating 230 is disposed about the central portion 217 of the elongate member 210. In some embodiments, the coating 230 can be disposed about a different portion for the elongate member, e.g., the proximal end portion 213 and/or the distal end portion 215. In some embodiments, the coating 230 can be disposed about substantially the entire length of the elongate member 210. In yet other embodiments, the coating 230 can be disposed about the fixation mechanism 220.

The coating 230 is formulated to release, elute, and/or convey at least one material (i.e., the releasate) into the body of the patient. Said another way, the coating 230 includes a releasate that can be released, eluted, and/or conveyed when the elongate member 210 is disposed in the body of the patient. In some embodiments, the coating 230 can be formulated to release the releasate when an electrical current is transmitted through a portion of the elongate member 210, e.g., from the proximal end portion 213 to the distal end portion 215. In other words, in some embodiments, transmission of the electrical current through the elongate member 210 enhances the release kinetics of the coating 230. In some embodiments, for example, the transmission of the electrical current can enhance the release kinetics of the coating 230 by increasing a localized temperature of the coating, altering the porosity of the coating 230, stimulating a catalyst contained within the coating 230, or the like. In this manner, a medical professional, the patient, or other user can control the release and/or the release rate of the releasate from the coating 230 by controlling transmission of the electrical current or stimulation through the elongate member 210. In other embodiments, the coating 230 can include a bioerodible polymer, of the types discussed herein, such that the releasate can be released, eluted and/or conveyed when a portion of the coating 230 erodes within the body.

The releasate can be at least one of a therapeutic agent, a conductive agent, and/or an insulative agent. The therapeutic agent can be, for example, any compound or mixture of compounds capable of modifying or modulating the function of at least one biological system. For example, the therapeutic agent can be configured to treat and/or alleviate at least one of infection, inflammation, pain, scar tissue formation, or other undesirable indication, which may be preexisting or which may arise due to implantation of the electrical conductor 200 in the body of the patient. The therapeutic agent can be, for example, an antimicrobial agent (including polysporin); an anti-inflammatory agent (including corticosteroid); and/or a pain reducer (including dibucaine, dibucaine free base, dibucaine HCl, and/or bupivacaine HCl).

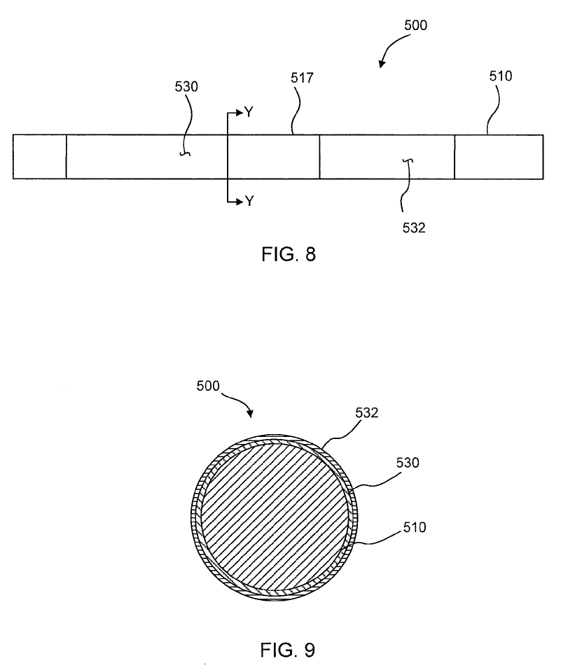

The conductive agent facilitates, or enhances, transmission of the electrical current from the electrode 212 to the target bodily tissue, e.g., after the conductive agent is released into the body of the patient. Similarly stated, the conductive agent can define and/or enhance a portion of a stimulation pathway within the bodily tissue along which the electrical current can travel. The conductive agent can be, for example, an electrically conductive medium, electrically conductive particles, or the like. An electrically conductive medium can be, for example, a polymer configured to release, elute, or otherwise deliver an electrically conductive particle or solution. The conductive agent can be or include a polymer that is more conductive the bodily tissue surrounding the electrical conductor 200. The polymer can include, for example, a complex of polyacid-poly(N-vinyl pyrrolidone), which has conductivity greater than about 0.04 S/m, and thus is more conductive that some subcutaneous tissues that have conductivity of about 0.04 S/m (i.e., with a stimulation frequency less than 100 Hz). In this manner, the coating 230 has a greater electrical conductivity than the body of the patient (e.g., the coating 230 has a greater electrical conductivity than the bodily tissue surrounding the electrical conductor 200). Release of the conductive agent into a body of a patient is described in more detail herein with respect to FIGS. 6 and 7.

The insulative agent enhances travel of the electrical current within the bodily tissue. The insulative agent is configured to prevent loss of and/or limit the attenuation of electrical current to (or help recover lost electrical current from) surrounding bodily tissue such that a greater portion of the electrical current reaches the target bodily tissue than would otherwise reach the target bodily tissue without the insulative agent. The insulative agent can be, for example, an insulative medium, insulative polymer, insulative particles, or the like. An electrically insulative medium can be, for example, a polymer configured to release, elute, or otherwise deliver an electrically insulative particle or solution. Release of the insulative agent into a body of a patient is described in more detail herein with respect to FIGS. 6 and 7.

The fixation mechanism 220 of the electrical conductor 200 is disposed on and/or coupled to the elongate member 210. As illustrated in FIG. 2, the fixation mechanism 220 is disposed about the distal end portion 215 of the elongate member 210. The fixation mechanism 220 is configured to retain the elongate member 210 within the body of the patient. Similarly stated, the fixation mechanism 220 is configured to limit rotational and/or translational movement of the elongate member 210 within the body of the patient. The fixation mechanism 220 includes tines 222, 222′. The tines 222, 222′ are movable between a collapsed position (FIG. 4) and an expanded position (FIG. 2). In the expanded position, the tines 222, 222′ are configured to engage bodily tissue in a manner to substantially retain the position of the elongate member 210 with respect to the bodily tissue and/or to substantially inhibit regression (e.g., movement in the proximal direction) of the elongate member 210 from the bodily tissue. The fixation mechanism 220 can be any suitable fixation mechanism of the types shown and described in U.S. Patent Application Attorney Docket No. BION-004/01US 307799-2091, entitled “Electrical Stimulation Lead with Bioerodible Anchors and Anchor Straps,” filed on Oct. 14, 2008, which is incorporated herein by reference in its entirety.

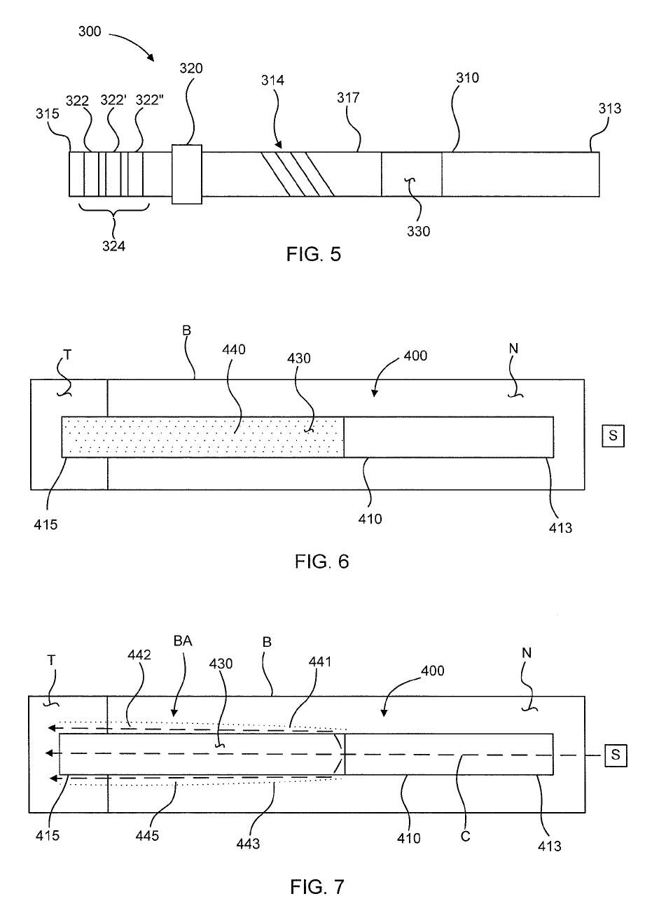

A medical implant 300 according to an embodiment is illustrated in FIG. 5. The medical implant 300 is configured to conduct (or transmit) an electrical current between an external stimulator (not shown) and a target bodily tissue (not shown) of a patient. The medical implant 300 includes an elongate member 310, a set of electrodes 324, and a fixation mechanism 320.

The elongate member 310 has a proximal end portion 313, a distal end portion 315 and a central portion 317 extending between the proximal end portion 313 and the distal end portion 315. The set of electrodes 324 is disposed on the distal end portion 315 of the elongate member 310.

The proximal end portion 313 of the medical implant 300 is configured to be in electrical communication with the external stimulator. For example, the proximal end portion 313 of the elongate member 310 can receive an electrical current from the stimulator via a mechanical connection, as described above. In another example, the proximal end portion 313 of the elongate member 310 can receive an electrical current from the bodily tissue of the patient. The electrical current is transmitted from the proximal end portion 313 to the distal end portion 315 of the elongate member 310 via the central portion 317. The electrical current is transmitted from the distal end portion 315 of the elongate member to the target bodily tissue by each electrode 322, 322′, 322″.

The elongate member 310 includes a core (not shown) and a coating 330. The coating 330 is disposed on at least a portion of the core of the elongate member 310. The coating 330 is formulated to elute, release and/or deliver a releasate into the body of the patient over a desired period of time. The releasate can be a material, such as a therapeutic agent, a conductive agent, and/or an insulative agent, as described above. The coating 330 can be formulated to deliver or release the releasate in a relatively short time period, such as, for example, several minutes or hours up to several weeks or months. In another example, the coating 330 can be formulated to deliver or release the releasate over a relatively long time period, such as at least several months, a year, or longer.

Release or elution of the releasate is determined by various factors, including, for example, the surface area of the portion of the elongate member 310 about which the coating 330 is disposed, the density of the coating 330 on the elongate member 310, the thickness of the coating 330, the concentration of the coating 330 on the elongate member 310, characteristics of the coating material (e.g., the porosity), and/or characteristics of the releasate. For example, characteristics of the releasate, such as composition, molecular weight, charge density, hydrophobicity, solubility of the releasate in the coating material, ratio of the releasate to the coating material, method of incorporation of the releasate into the coating material, and releasate volume, can affect the rate or release or elution of the releasate. Thus, any of the foregoing factors can be adjusted to formulate the coating to release the releasate over the predetermined period of time.

In some embodiments, the coating 330 includes a bioerodible material, e.g., a bioerodible polymer. As used herein, bioerodible material is a material capable of being degraded, disassembled, and/or digested by action of a biological environment (including the action of living organisms) and/or in response to a change in physiological pH, a change in temperature, and/or electrical stimulation. In this manner, the bioerodible polymer is configured to deliver, release and/or elute the releasate into the surrounding bodily tissue as the polymer bioerodes in the body of the patient. For example, the releasate can be an electrically insulative medium embedded in the polymer that is configured to be released over time as the polymer erodes. In another example, the coating includes an electrically conductive bioerodible polymer configured to be released into the bodily tissue as the coating and/or polymer bioerodes.

In some embodiments, the coating 330 and/or any of the coatings described herein can include or be formed of at least one of the following bioerodible polymers (or a form of at least one of the following polymers): polydioxanone; aliphatic or other polyesters (including polycaprolactone and polyglycolide); modified polysaccharides and other natural polymers (including cellulose acetate butyrate); poly(ethylene glycol) based polymers (including poly(ethylene)oxide); poly(ethylene glycol)-poly(propylene glycol) based polymers (including poly(ethylene glycol-ran-propylene glycol)); poly(vinyl alcohol) and copolymers (including poly(vinyl alcohol-co-ethylene)); hydrogels or other crosslinked polymers (including poly(N-isopropylacrylamide)); hydrophilic polymers (including polyvinylpyrrolidone); hydrophobic polymers (including poly(4-vinylphenol)); a crosslinker (including divinylbenzene); or the like; or a combination thereof.

Other suitable bioerodible polymers include poly[bis(p-carboxyphenoxy)propane anhydride] (pCPP):sebacic acid (SA), polylactic acid, polyanhydride, polycaprolactone, and polyglycolic acid, or the like, or a combination thereof. Other suitable bioerodible polymers are described or discussed in U.S. Pat. No. 5,030,457 to Ng et al., U.S. Pat. Nos. 5,939,453 and 5,968,543 to Heller et al., U.S. Pat. No. 6,153,664 to Wise et al., and U.S. Pat. No. 6,304,786 to Heil Jr. et al, each of which are incorporated herein by reference.

In some embodiments, the conductive agent can include an electrically conductive bioerodible polymer. The electrically conductive bioerodible polymer can include a complex including polyacid-poly(N-vinyl pyrrolidone), polypyrrole, polyvinylpyrrolidone, or any other suitable polymer, such as a polymer described in U.S. Pat. Nos. 3,494,907 and 3,563,968 to Merijan et al. and U.S. Pat. No. 4,702,732 to Powers et al, each of which is hereby incorporated by reference.

In embodiments in which the coating 330 and/or any other coating described herein is constructed of a bioerodible polymer, a releasate can be released from the coating as the polymer erodes. For example, in one embodiment, an insulative agent can be released into the body of the patient when at least a portion of the bioerodible polymer erodes, which can reduce the need for inclusion of an insulative backing on the medical implant. In another example, a therapeutic agent can be released into bodily tissue surrounding the implanted medical implant when at least a portion of the polymer erodes. In yet another example, a conductive agent can be released into bodily tissue surrounding the implanted medical implant when at least a portion of the polymer erodes. In some embodiments, the agent is included or disposed within a matrix of the polymer.

In some embodiments, the coating 330 can be formulated to release the releasate into the body of the patient in a controlled or sustained manner. For example, in some embodiments, the coating is configured to bioerode in a time-released manner. In this manner, the releasate (e.g., at least one of the therapeutic agent, the insulative agent, or the conductive agent) can enhance the effectiveness of the medical implant over a period of time, for example one year, depending on the indicated use of the medical implant. In this manner, the releasate (e.g., a therapeutic agent) can continuously manage pain or inflammation resulting from the presence of the medical implant in the body of the patient.

In another example, the coating 330 can be formulated to controllably release the releasate by at least one of polymer diffusion, dispersion, osmosis, polymer swelling, chemical control, dissolution, or active transportation by an electrical field. In yet another example, the coating 330 can be formulated to controllably release the releasate into the body of the patient in accordance with release kinetics based on laws of dispersion or Fickian diffusion. In still another example, the coating 330 can be formulated to controllably release the releasate by another suitable method of controlled-release delivery, such as a dissolution-controlled system that combines polymer swelling and slow macromolecular chain disentanglement to cause the controlled release of the releasate.

In some embodiments, the coating 330 is formulated to release the releasate in a non-continuous manner. For example, in some embodiments, the coating 330 can be formulated to release the releasate in at least one bolus (e.g., when the coating is exposed to the body of the patient). In other embodiments, the coating is formulated to release the releasate in multiple bolus. In yet other embodiments, the coating 330 can be formulated to release the releasate at a first rate for a first period of time and a second rate at for a second period of time.

The elongate member 310 defines a set of grooves 314 that extend obliquely to the longitudinal axis L. The set of grooves 314 are configured to retain the elongate member 310 within the body of the patient. For example, the set of grooves 314 is configured to prevent regression of the elongate member 310 within the bodily tissue, as discussed above. In some embodiments, the set of grooves 314 is defined by the core of the elongate member 310. In some embodiments, the set of grooves 314 is defined by the coating 330 of the elongate member 310.

FIGS. 6 and 7 illustrate a medical implant 400 disposed within a body at a first period of time, and a second period of time later than the first period of time, respectively. The medical implant 400 includes an elongate member 410 and a coating 430. The elongate member 410 has a proximal end portion 413, a distal end portion 415, and a central portion 417 extending between the proximal end portion 413 and the distal end portion 415. The coating 430 is disposed on a portion of the elongate member 410, e.g. the central portion 417 and/or the distal end portion 415. As shown in FIGS. 6-7, the medical implant 400 can be disposed within a body B such that the distal end portion 415 is adjacent a target bodily tissue T. The elongate member 410 is configured to deliver an electrical current C (shown in dashed lines) from an external source S (e.g., a stimulator) to the target bodily tissue T by conveying the electrical current C from the proximal end portion 413 to the distal end portion 415, as described above.

The coating 430 is formulated to release a releasate 440 into the body of the patient. The releasate 440 can be any suitable material, as described herein. After release from the coating 430, the releasate 440 is configured to form a barrier (shown in FIG. 7) disposed at least partially about the elongate member 410. Similarly stated, after the medical implant 400 has been disposed within the body for the second period of time (e.g., FIG. 7), at least a portion of the releasate 440 substantially surrounds the elongate member 410.

In some embodiments, for example, the releasate 440 includes an insulative agent of the types shown and described herein. The insulative agent 440 is configured to prevent loss of electrical current C to (or help recover lost electrical current from) surrounding bodily tissue N. More specifically, when the insulative agent 441 is within the coating 430, the insulative agent 441 is configured to prevent loss, or attenuation, of electrical current C from the medical implant 400 to the surrounding bodily tissue N. After the insulative agent 441 is released from the coating 430, the insulative agent 441 is configured to form an insulative barrier BA (shown in FIG. 7) disposed at least partially about the apparatus 400. Similarly stated, after the insulative agent 441 is released from the coating 430, the insulative agent 441 can define and/or enhance a stimulation pathway 442 within the body, through which the current C can be conveyed. In this manner, for example, the insulative agent 441 can limit attenuation of the electrical current to non-target bodily tissue N. In this manner, the insulative agent 441 is also configured to direct the electrical current C to the target bodily tissue T, for example, along a pathway 442 formed between the released insulative agent 441 and an outer surface of the elongate member 410. Thus, the insulative agent 441 is configured to increase the transfer of the electrical current C to the target bodily tissue T.

In another example, the releasate 440 can include a conductive agent 443 of the types shown and described herein. After the conductive agent 443 is released from the coating 430, the conductive agent 443 can define and/or enhance the stimulation pathway 442 within the body, through which the current C can be conveyed. Moreover, the released conductive agent 443 can form a second electrical pathway 445 leading from an area proximate to an outer surface of the elongate member 410 to the target bodily tissue T. Thus, at least a portion of electrical current C can be directed by the conductive agent towards the target bodily tissue T. The coating 430 can be formulated to release or elute the releasate 440 into the body of the patient in any manner described herein.

Although certain medical implants have been described herein as including a single coating, in other embodiments, a medical implant can include more than one coating. For example, as illustrated in FIGS. 8-9, a medical implant 500 according to an embodiment includes an elongate member 510 having a first coating 530 (or a first layer of coating) and a second coating 532 (or a second layer of coating) different than the first coating 530. As illustrated in FIG. 9, the first coating 530 and the second coating 532 overlap on a portion of the elongate member 510. Although the first coating 530 and the second coating 532 are illustrated in FIG. 9 as overlapping at a central portion 517 of the elongate member 510, in other embodiments, the first coating and the second coating can be disposed on and/or overlapping at a different portion of the elongate member. For example, in another embodiment, the first coating and the second coating can each be formed on or applied to a proximal end portion of an elongate member.

Although the medical implant 500 is shown and described as including an overlapping first coating 530 and second coating 532, in other embodiments, a medical implant includes an elongate member having a first coating formed on a first portion of the elongate member (e.g., an central portion) and a second coating different than the first coating formed on a second portion of the elongate member different than the first portion (e.g., a distal end portion). In some embodiments, a portion of the medical implant can have a different number of coatings than the number of coatings disposed on a different portion of the medical implant, or no coating at all.

The first coating 530 formulated to release a first releasate (e.g., a first therapeutic agent, a first conductive agent, and/or a first insulative agent) and the second coating 532 is formulated to release a second releasate (e.g., a second therapeutic agent, a second conductive agent, and/or a second insulative agent). The first coating 530 and the second coating 532 can be formulated to release any suitable combination of releasate (e.g., any suitable combination of a therapeutic agent, a conductive agent, and/or an insulative agent). For example, the first coating 530 can be formulated to release a therapeutic agent configured to treat pain and the second coating 532 can be formulated to release a therapeutic agent configured to treat infection. In another example, the first coating 530 can be formulated to release a therapeutic agent configured to treat inflammation and the second coating 532 can be formulated to release an electrically conductive medium configured to the surrounding bodily tissue. In still another example, the first coating 530 can be formulated to release an insulative agent and the second coating 532 can be formulated to release a therapeutic agent. Each coating (or layer of coating) 530, 532 can be formulated to release its respective releasate in any manner described herein. For example, in some embodiments, the first coating 530 can be formulated to release a therapeutic agent in at least one bolus into the body of the patient, such as when the first layer of coating is exposed to the body of the patient, and the second coating 532 can be formulated to release an insulative agent in response to an electrical stimulation.

Although the medical implant 500 is illustrated as including an elongate member 510 having two coatings (or two layers of coating) 530, 532, in other embodiments, a medical implant can include an elongate member having a multi-layered coating that includes three layers. In some embodiments, for example, the first layer of coating can be, formulated to release a first releasate into the body of the patient. The second layer of coating can be formulated to bioerode within the body of the patient. The second layer of coating contains no releasate, but exposes the third layer of coating as the second layer erodes. The third layer of coating can be formulated to release a second releasate into the body of the patient. The first releasate and the second releasate can be different or similar releasates.

Although the medical implant 500 is shown as including two distinct coatings, in other embodiments, a medical implant can include a coating having multiple regions. Each region of the coating can be disposed on a portion of the elongate member different than a portion of the elongate member on which another region is disposed. In some embodiments, for example, the coating can be discontinuous and/or non-contiguous along a length and/or a circumference of the elongate member. For example, in one embodiment, a first region of the coating is disposed on a proximal end portion of the medical implant and a second region of the coating is disposed on a distal end portion of the medical implant. At least one region of the coating is formulated to release a releasate. For example, in some embodiments, the first region of the coating is formulated to release a first releasate and the second region of the coating is formulated to release a second releasate different than the first releasate.

The concentration and/or amount of a releasate included within a coating can be spatially variable along the length and/or circumference of the medical implant. For example, in some embodiments, the concentration and/or amount of a releasate in one region of the coating can vary from the concentration or amount of a releasate in a different region. In another example, the concentration and/or amount of a releasate in one region of the coating can vary from the concentration and/or amount of a different releasate in the same region or in a different region. In this manner, for example, the medical implant can be configured to release a first concentration of a releasate to bodily tissue proximate to a first region and a second concentration of a releasate to bodily tissue proximate to a second region. The magnitude of the first concentration of the releasate can be associated with the location of the bodily tissue on which the releasate is to act and/or the desired timing of the release of the releasate from the coating. In some embodiments, for example, a medical implant includes a coating having a first region formulated to release a releasate in one bolus and having a second region formulated to controllably or sustainably release the releasate over a desired period of time. In another example, a medical implant includes a coating having a first region formulated to release a first releasate (e.g., a pain relieving therapeutic agent) and a second region formulated to release a second releasate (e.g., an anti-inflammatory therapeutic agent) different than the first releasate.

In some embodiments, a medical implant includes a coating having a first region formulated to release an electrically conductive medium or other conductive agent and having a second region formulated to release a therapeutic agent. For example, the coating can have a first region disposed on a proximal end portion of the medical implant and that is formulated to release the electrically conductive medium over a long period of time, and a second region disposed on a distal end portion of the medical implant and that is formulated to release a therapeutic agent over a short period of time, such as to help prevent inflammation, scar tissue, and/or pain for the period following insertion of the medical implant in the body of the patient. Although the coating is described herein as including two different regions or being disposed on two different portions of the electrical conductor, in other embodiments, the coating can include one, three, four or more regions disposed on one or more portions of the medical implant.

Each portion of the medical implant described herein can be constructed of any suitable material. For example, in some embodiments, at least one of the elongate member, the coating, or the insulative layer (e.g., insulative layer 216) includes or is constructed of a polymer, for example a polymer selected from a class of plastics that adheres to the ISO 10993 standards for prolonged and permanent implantation in a body of a patient. In another example, the polymer can be a Class VI plastic as identified by the United States Pharmacopeia.

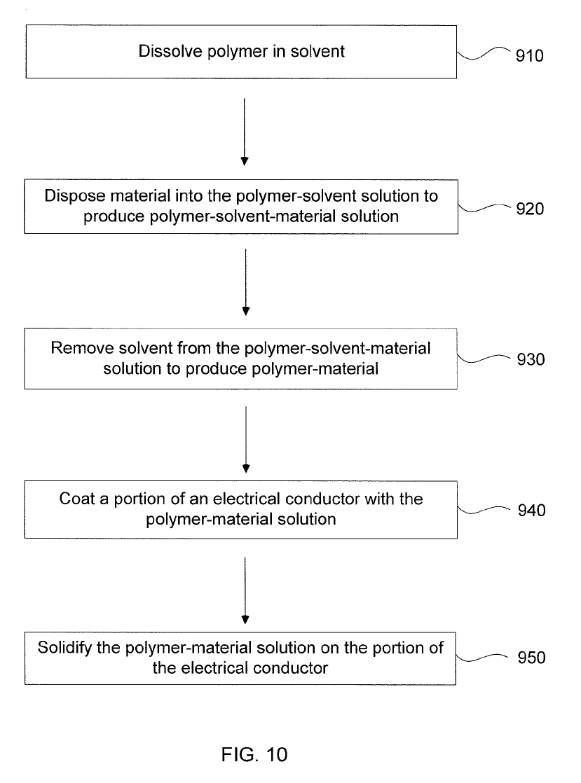

FIG. 10 illustrates a flowchart of a method for applying or disposing a coating (e.g., coating 230, coating 330, coating 332) on an outer surface of a medical implant (e.g., electrical conductor 200, medical implant 300). At activity 910, a polymer is dissolved in a solvent, thereby producing a polymer-solvent solution. In some embodiments, the solvent includes chloroform, methanol, or a combination of the foregoing. In other embodiments, the solvent is any solvent suitable for dissolving the selected or provided polymer. The solvent can be selected from a group of solvents identified as acceptable according to ISO standards and the United States Pharmacopeia, such as a solvent that is at least a Class II or III solvent according to the United States Pharmacopeia.

Optionally, the polymer can be selected based on the time period within which a material (i.e., releasate) will be released from the resulting coating. Said another way, a polymer is selected or provided based on whether the resulting coating is configured for short term release of a material, or whether the resulting coating is configured for long term release of the material. The material can include at least one of a therapeutic agent, a conductive agent, or an insulative agent. The material can be configured to be eluted or otherwise released into the body of the patient, as described herein.

At activity 920, the at least one material to be eluted or otherwise released into the body of the patient is disposed into the polymer-solvent solution. Any known process for incorporating the therapeutic material into the polymer may be used, for example, as disclosed in U.S. Pat. No. 5,030,457 to Ng et al. and U.S. Pat. No. 5,939,453 to Heller et al, each of which is incorporated herein by reference. Optionally, disposing the material into the polymer-solvent solution can include dissolving the material in the solution. Also optionally, an amount of the material can be incorporated into the polymer-solvent solution to achieve a desired ratio of material to the polymer-solvent solution. For example, an amount of the material can be dissolved in the polymer-solvent solution to create a polymer-solvent-material solution having a desired concentration of material in the solution.

In some embodiments, the method optionally includes producing a mathematical model of the elution, release, erosion and/or dissolution of the polymer and/or material when the resulting electrical conductor is disposed within the body. For example, in some embodiments the method includes comparing the modeled elution, release, erosion and/or dissolution of the polymer and/or releasate to known in vitro and in vivo performance. In this manner, the elution, release, erosion and/or dissolution of the polymers and/or the releasate can be optimized. For example, in some embodiments, the ratios of polymer to material and/or solvent can be adjusted based on the mathematical model.

At activity 930, the solvent is at least partially removed from the polymer-solvent-material solution. The solvent can be removed in any suitable known manner, for example, by evaporation. In this manner, the polymer-solvent-material solution is concentrated by evaporation to remove the solvent and to retain the polymer-material solution. The solvent can be, for example, evaporated using a standard evaporator, and the polymer-material mixture can be collected from or retained in a bottom of an evaporator flask.

At activity 940, a portion of an electrical conductor, or other medical implant, is coated with the polymer-material solution. The coating can be applied to the electrical conductor in any suitable known manner. For example, the electrical conductor can be dipped or otherwise immersed in the polymer-material solution. In this manner, the electrical conductor can be disposed within the solution for a desired period of time, e.g. a period of time associated with a desired thickness of the coating. In another example, the coating can be applied to the electrical conductor by a physical vapor deposition. For example, the coating can be applied by sputter deposition in which the polymer-material solution is sputtered onto the desired portion of the electrical conductor. At activity 950, the polymer-material solution is solidified on the electrical conductor. In this manner, a coating is formed on the electrical conductor.

Coating and solidifying the polymer-material solution on the electrical conductor can be individually and/or alternatively repeated until a desired amount and/or thickness of coating is disposed on the electrical conductor. Optionally, a desired length of time for release of the material within the body of the patient can be identified. A coating thickness associated with the identified length of time can also be identified. The coating and the solidifying can be repeated until the identified coating thickness is achieved on the portion of the electrical conductor. For example, the coating and solidifying the coating can be performed multiple times, such when a long term release of the therapeutic agent is desired.

The medical implants described herein are suitable for a variety of applications. For example, in one procedure, at least a portion of a medical implant (e.g., electrical conductor 200) is implanted within a body of a patient. An electrical current is transmitted to the electrical conductor 200. At least one releasate, e.g., a therapeutic agent, a conductive agent, an insulative agent, or the like, is released from the coating 230 in response to the transmission of the electrical current to the electrical conductor 200. In some embodiments, the transmitting the electrical current can include transmitting an electrical current sufficient to facilitate release from the coating of the releasate in at least one bolus. In some embodiments, the transmitting the electrical current can include transmitting an electrical current sufficient to facilitate at least partial erosion of a polymer of which the coating is formulated such that the releasate is released from the coating in response to erosion of the polymer of the coating. In some embodiments, at least one biocompatible agent can be at least partially eluted over time into the body of the patient. In a procedure in which the patient requires additional delivery of a releasate after the releasate has been released from the coating of the electrical conductor, the electrical conductor can be removed from the body of the patient and a new electrical conductor can be reinserted into the body of the patient.

While various embodiments have been described above, it should be understood that they have been presented by way of example only, and not limitation. Although various embodiments have been described as having particular features and/or combinations of components, other embodiments are possible having a combination of any features and/or components from any of embodiments as discussed above.

Where methods described above indicate certain events occurring in certain order, the ordering of certain events may be modified. Additionally, certain of the events may be performed concurrently in a parallel process when possible, as well as performed sequentially as described above. Furthermore, although methods are described above as including certain events, any events disclosed with respect to one method may be performed in a different method according to the invention. Thus, the breadth and scope should not be limited by any of the above-described embodiments. While the invention has been particularly shown and described with reference to specific embodiments thereof, it will be understood that various changes in form and details may be made.

For example, although certain medical implants (e.g., electrical conductor 200, medical implant 300) illustrated and described herein include a set of grooves defined by an central portion of the medical implant, in other embodiments, the set of grooves can be defined by a different portion of the medical implant. For example, a set of grooves can be defined by an insulative layer disposed over a proximal end portion of an elongate member, over a distal end portion of the elongate member, and/or a portion of the elongate member therebetween. In another example, the set of grooves can be defined by the elongate member.

Although certain medical implants illustrated and described herein (e.g., electrical conductor 200, medical implant 300) include a set of four grooves (e.g. set of grooves 214, set of grooves 314) defined by the elongate member (e.g., elongate member 210, elongate member 310), in other embodiments, an elongate member can include any suitable number of grooves, for example, one, two, three, or more, or none. In another embodiment, a portion of the medical implant can define a set of grooves extending obliquely and a set of grooves extending circumferentially about the elongate member and orthogonally to the longitudinal axis of the elongate member.

In still other embodiments, an elongate member can be differently configured to retain the elongate member within the bodily tissue. For example, an elongate member can have a textured portion configured to engage the bodily tissue.

Although elongate members illustrated and described herein include electrodes disposed on the distal end portion of the elongate members, in other embodiments, an electrode can be disposed on a different portion of an elongate member, for example an central portion or a proximal end portion of the elongate member.

Although elongate members illustrated and described herein include one or three electrodes, in other embodiments, an elongate member can include any suitable number of electrodes, for example two, four, or more electrodes.

Although the insulative layer 216 has been illustrated and described as being disposed over an outer surface of the elongate member 210, in other embodiments, the insulative layer can be disposed on a portion of the elongate member. For example, in some embodiments, the insulative layer is disposed on at least one of the proximal end portion, the distal end portion, and/or the central portion.

Although the insulative layer 216 has been described herein as being constructed of a polymer, in other embodiments, the insulative layer can be construction of another material or combination of materials suitable for implantation into a body of a patient. Such material can include, for example, ceramics.

Although the insulative layer 216 has been illustrated and described as being disposed on a portion of the core 211 of the elongate member 210, in other embodiments, the insulative layer can be disposed about substantially the entire length of the core of the elongate member.

Although the coatings are shown and described herein as including a releasate material therein, in some embodiments, a medical implant can include a coating having a reservoir and/or membrane system. At least one therapeutic agent, conductive agent, and/or insulative agent can be embedded in the reservoir or membrane system. For example, the coating can include a reservoir system configured to release a therapeutic agent after eluting a membrane which separates the layers of the coating. In another embodiment, the therapeutic agent is embedded in a matrix system. For example, the coating can include a matrix system that is modeled based on porosity (or number of open pores), the nature of the loading mechanism as dissolved or dispersed, and the solubility limits in water.

Although certain medical implants have been described herein as including a coating (e.g., coating 130, coating 230, coating 330) formulated to release a releasate that is a therapeutic agent, a conductive agent, and/or an insulative agent, in other embodiments, an apparatus includes a coating formulated to release any suitable material.

Although certain coatings (e.g., coating 230, coating 430) have been illustrated and described herein as being circumferentially disposed about the core and/or the insulative layer of the elongate member (e.g., elongate member 210, elongate member 410), in other embodiments, the coating can be disposed about only a portion of the circumference of the core and/or the insulative layer of the elongate member.

Although certain medical implants have been described herein as including a multi-layered coating that has two or three layers, in other embodiments, a medical implant can include an elongate member having a multi-layered coating with any suitable number of layers. For example, in other embodiments, a multi-layered coating can include four, five, or more layers.

Although the fixation mechanism 220 has been illustrated and described as including two tines 222, 222′, in other embodiments, the fixation mechanism can include any suitable number of tines, for example, one, three, four, or more.

Although certain medical implants have been illustrated and described herein as being configured to receive an electrical current from a external source, in other embodiments, the apparatus can be configured to receive an electrical input from an internal source. For example, a proximal end portion of the apparatus can be configured to receive an electrical input from a source disposed within an elongate member of the medical implant.

What is claimed is:

1. A medical implant, comprising:
an elongate member including a proximal end portion and a distal end portion, the proximal end portion configured to receive an electrical current from a current source, the elongate member configured to transmit the electrical current from the proximal end portion to the distal end portion, at least a portion of the elongate member configured to be disposed within a body of a patient;
an electrode coupled to the distal end portion of the elongate member, the electrode configured to transmit a portion of the electrical current from the distal end portion of the elongate member to a target bodily tissue; and
a coating disposed on at least a portion of the elongate member, the coating formulated to release at least one of a therapeutic agent, a conductive agent, or an insulative agent into the body of the patient in response to the electrical current being transmitted from the proximal end portion to the distal end portion of the elongate member.

2. The apparatus of claim 1, wherein the coating includes a bioerodible polymer formulated to release the at least one of the therapeutic agent, the conductive agent, or the insulative agent when the bioerodible polymer erodes within the body of the patient.

3. The apparatus of claim 1, wherein the at least one of the therapeutic agent, the conductive agent, or the insulative agent is controllably released by at least one of polymer diffusion, dispersion, osmosis, polymer swelling, chemical control, dissolution, or active transportation by an electrical field.

4. The apparatus of claim 1, wherein the coating is formulated to release substantially all of the at least one of the therapeutic agent, the conductive agent, or the insulative agent within a predetermined time after the portion of the elongate member is disposed within the body of the patient.

5. The apparatus of claim 1, wherein the coating is formulated to release the at least one of the therapeutic agent, the conductive agent, or the insulative agent at a predetermined rate of release when the portion of the elongate member is disposed within the body of the patient.

6. The apparatus of claim 1, wherein the at least one of the therapeutic agent, the conductive agent, or the insulative agent is configured to be released in at least one bolus.

7. The apparatus of claim 1, wherein the coating has a greater electrical conductivity than a bodily tissue of the patient in which the elongate member is configured to be disposed.

8. The apparatus of claim 1, wherein the insulative agent is configured to prevent loss of the electrical current to non-target bodily tissue.

9. The apparatus of claim 1, wherein the insulative agent is configured to direct the electrical current to the target bodily tissue.

10. The apparatus of claim 1, wherein the therapeutic agent is a first therapeutic agent, the conductive agent is a first conductive agent, and the insulative agent is a first insulative agent, the coating has a first layer including the at least one of the first therapeutic agent, the first conductive agent, or the first insulative agent, the coating has a second layer including at least one of a second therapeutic agent, a second conductive agent, or a second insulative agent.

11. The apparatus of claim 1, wherein the elongate member includes a retention member configured to limit movement of the elongate member within the body of the patient.

12. The apparatus of claim 1, wherein the elongate member includes at least one groove configured to prevent regression of the elongate member within bodily tissue.

13. The apparatus of claim 1, wherein the elongate member includes an insulative layer.

14. A method, comprising:
dissolving a polymer into a solvent to produce a polymer-solvent solution;
disposing a material into the polymer-solvent solution to produce a polymer-solvent-material solution;
removing the solvent from the polymer-solvent-material solution to produce a polymer-material solution;
coating a portion of an electrical conductor with the polymer-material solution; and
solidifying the polymer-material solution on the portion of the electrical conductor.

15. The method of claim 14, wherein the polymer is formulated to be bioerodible when disposed within a body of a patient.

16. The method of claim 14, further comprising:
selecting a polymer based on a desired time period for release of the material from the coating into a body of a patient.

17. The method of claim 14, wherein the solvent includes at least one of chloroform or methanol.

18. The method of claim 14, wherein the material includes at least one of a therapeutic agent, a conductive agent, or an insulative agent.

19. The method of claim 14, wherein the disposing includes disposing an amount of the material into the polymer-solvent solution such that a ratio of material to the polymer-solvent solution is within a predetermined range.

20. The method of claim 14, wherein the removing includes evaporating a portion of the solvent from the polymer-solvent-material solution.

21. The method of claim 14, wherein the coating includes inserting the portion of the electrical conductor into the polymer-material solution for a period of time.

22. The method of claim 14, further comprising:
repeating the coating and the solidifying until a desired amount of polymer-material solution is disposed on the portion of the electrical conductor.

23. The method of claim 14, further comprising:
identifying a period of time within which the material is to be released within a body of a patient after the electrical conductor is disposed within the body of the patient;
associating a coating thickness with the time period; and
repeating the coating and the solidifying until the coating thickness is achieved on the portion of the electrical conductor.

24. The method of claim 14, wherein the coating includes sputtering the polymer-material solution onto the portion of the electrical conductor.

25. A method, comprising:
implanting at least a portion of an electrical conductor within a body of a patient; and
transmitting an electrical current to the electrical conductor, at least one of a therapeutic agent, a conductive agent, or an insulative agent being released from a coating on an exterior portion of the electrical conductor in response to the transmitting of the electrical current to the electrical conductor.

26. The method of claim 25, wherein the therapeutic agent is formulated to include at least one of an antimicrobial, an anti-inflammatory, or a pain reliever.

27. The method of claim 25, wherein the conductive agent is configured to conduct an electrical current within a bodily tissue.

28. The method of claim 25, wherein the conductive agent has a conductivity higher than a conductivity of a bodily tissue proximate to the electrical conductor.

29. The method of claim 25, wherein the insulative agent is configured to prevent attenuation of the electrical current to non-target bodily tissue.

30. The method of claim 25, wherein the transmitting the electrical current includes transmitting an electrical current sufficient to facilitate release from the coating of the at least one of the therapeutic agent, the conductive agent, or the insulative agent in at least one bolus.

31. The method of claim 25, wherein the transmitting the electrical current includes transmitting an electrical current sufficient to facilitate erosion of at least a portion of a polymer disposed within the coating, the at least one of the therapeutic agent, the conductive agent, or the insulative agent being released from the coating in response to the erosion of the portion of the polymer.

32. A medical implant, comprising:
an elongate member including a proximal end portion and a distal end portion, the proximal end portion configured to receive an electrical current from a current source, the elongate member configured to transmit the electrical current from the proximal end portion to the distal end portion, at least a portion of the elongate member configured to be disposed within a body of a patient;
an electrode coupled to the distal end portion of the elongate member, the electrode configured to transmit a portion of the electrical current from the distal end portion of the elongate member to a target bodily tissue; and
a coating disposed on at least a portion of the elongate member, the coating formulated to release at least one of a conductive agent or an insulative agent into the body of the patient.
